(19) United States
(12) Patent Application Publication
(10) Pub. No.: US 2012/0253203 A1
(43) Pub. Date: Oct. 4, 2012

(54) METHOD OF VIEWING A SURFACE

(76) Inventors: Nigel Weston, Bracknell (GB); Darren Allen Glass, Deerfield Township, OH (US); Thomas Arthur Sturgis, Mason, OH (US); Helen Rochelle Kemp, Glendale, OH (US); Oliver David Oglesby, Newbury (GB); Keith Edward Paskins, Geneva (CH); Jamie-Lee Helen Weeks, Reading (GB)

(21) Appl. No.: 13/433,387

(22) Filed: Mar. 29, 2012

Related U.S. Application Data
(60) Provisional application No. 61/469,389, filed on Mar. 30, 2011.

(51) Int. Cl. A61B 6/00

(52) U.S. Cl. 600/476

(57) ABSTRACT

A method of viewing a surface comprising taking at least a first image from a first position and a second image from a second position to allow for comparative analysis and diagnosis of the surface.

### CROSS REFERENCE

This application claims the benefit of U.S. Provisional Application Ser. No. 61/469,389, filed on Mar. 30, 2011, the contents of which are incorporated herein by reference.

### FIELD OF THE INVENTION

This invention generally relates to imaging of surfaces, preferably skin having hair on humans or beasts.

### BACKGROUND OF THE INVENTION

Pseudofolliculitis barbae (PFB) is a common inflammatory skin disease, particularly among African American men. This condition has been reported to occur when an ingrown facial hair triggers a foreign body inflammatory response and is characterized by the development of lesions, papules and pustules. Lesion formation and skin irritation associated with PFB can lead to men decreasing their shave frequency to 2-3 times a week. However, a low shave frequency can in turn increase the likelihood of lesion formation as the hairs are given a chance to grow long enough to continually re-enter the skin. Daily shaving minimizes hair length and can thus help reducing the opportunity of lesion formation via ingrown hairs. However, daily shaving is perceived by some who have this condition as being uncomfortable. Moreover, it has been reported that many men believe that daily shaving with a multi-blade razor can exacerbate ingrown facial hair, ultimately leading to PFB.

Another common occurrence of skin irritations related to hair growth is where hairs growing from a portion of skin grow out of the skin at an angle or otherwise become at least partially obstructed and cause skin irritations. Acne is another common skin irritation that can be problematic for some.

There have been many attempts to treat or minimize the occurrence of PFB and related skin conditions by using different shaving tools as well as skin care compositions. See, e.g., U.S. Pat. Nos. 7,404,949; 5,853,709 4,944,939; 4,775,530; and 4,228,163. Additional reports describing PFB and other related skin conditions have been described in KOSMET and MEDLINE reports, such as: Zupkowsky, Pamela et al., International Journal of Cosmetic Science, 2009, vol. 31, pg. 312; Garcia-Zuazaga Jorge, Military Medicine, July 2003, Vol. 168, No. 7, pg. 561-4; and Crutchfield C E 3^{rd}, Cutis; Cutaneous Medicine for the Practitioner, June 1998, Vol. 61, No. 6, pg. 351-6.

Many forms of viewing tools and microscopes are known in the art. Examples of some of these devices are available in U.S. Patent Pub. No. 2011/0058030, 2006/0034485, and 2004/0201694; U.S. Pat. Nos. 7,859,749, 5,719,700.

Despite all the attempts to treat and minimize occurrences of these types of skin conditions and the many types of viewing tools which are generally available in the field of optics and skin care, the diagnosis of these conditions is commonly done visually and at random. To better understand and diagnose the occurrence of these types of skin conditions and symptoms, a new method is needed which can provide more consistent diagnosis and identification of various skin and hair growth conditions.

### SUMMARY OF THE INVENTION

One aspect of the present invention provides for a method of viewing a portion of surface, optionally having at least one hair, comprising: taking a first image of a focal point of said portion of skin from a first position, taking a second image of said portion of skin from a second position, wherein an angle between the first position to the focal point and the second position and the focal point is at least about 15°, and wherein the first image is taken at the same focal length from the focal point as the second image. In another aspect the method further comprises an analysis step, as well as an optional recommendation step.

In one embodiment, the step of imaging is performed using a digital microscope having at least one reflective member, a camera or video camera having one or more lenses which are focused on the same focal point.

### BRIEF DESCRIPTION OF THE DRAWINGS

FIGS. 1-8 are elevated perspective views of various embodiments of the present invention wherein a surface is viewed from one or more positions. Shown in each of these figures is a three dimensional Cartesian coordinate system with X, Y and Z axes. FIG. 9 is a perspective view of one possible embodiment of a digital microscope suitable for use in accordance with the method of the present invention.

### DETAILED DESCRIPTION OF THE INVENTION

The present invention relates to a method of imaging a surface, preferably skin having one or more hairs or hair follicles. It has been found that this method of imaging can be particularly useful for diagnosing various skin conditions, including but not limited to one or more of PFB, abrasions, obstructed hairs, acne, and the other conditions described above. Of course, in addition to the identification of the occurrence of these types of conditions, the present method can also be used to detect the relative absence of such conditions or symptoms so a subject can be informed of the relative condition of their skin.

Without intending to be bound by theory, it is believed that the present invention allows for multi-angled imaging of a surface to allow for a comprehensive viewing which can be used for subsequent analysis and diagnosis into a skin or hair condition. This analysis can then be used to identify a treatment regimen or assignment of a relative skin/hair health measure. The terms viewing and imaging are used throughout the application. Viewing is intended to mean the general act of observing an object. Imaging is intended to be the actual capturing of the image via any known recordable means (including but not limited to digital or film photography, x-ray, thermographic imaging, visual inspection, and so forth).

In one embodiment, the imaging can also be done via video. In such an embodiment, the stereo imaging would be stereo video capture.

1. Viewing Step

In one embodiment of the present inventions, the method of comprises a step of viewing said portion of skin comprising at least one hair, said viewing step comprising: taking a first image of a focal point of said portion of skin from a first position, taking a second image of said portion of skin from a second position. In one embodiment, the method comprises taking a third or further subsequent image. In another embodiment, multiple shots of one or more of the images can be taken, for example the step of taking a first image can include taking two or more shots of the same image. It is believed that taking multiple shots of any of the images can be preferable should one or more of the shots be undesirably blurred or obscured during the viewing steps.

The focal point, as defined herein, refers to is the point in space where parallel light rays meet after passing through the lens of a viewing position. Since light can pass through a lens in either direction, a lens has two focal points—one on each side. The focal point in this invention refers to the position on the surface or in the vicinity of the surface which the image is taken. Preferably, the image(s) are taken while the surface is in focus and not blurry. The distance in air from the lens principal plane to the focus is called the focal length. In one embodiment, the focal point is a discrete point on the surface. In another embodiment, the focal point is chosen such that it is above or below the plane of the surface. Preferably the focal point is on the surface. The focus point, can however, rest above the surface, particularly if the focal point is chosen as a position along the length of a hair. As such, in one embodiment, the focal point is from the surface to a distance of about 5 cm above said surface, or to a distance of about 3 cm above said surface, or to a distance of about 1 cm above said surface.

In one embodiment, the surface is viewed within a predetermined amount of time from having been shaved. In one embodiment the predetermined amount of time is from about 24 to about 72 hours of having been shaved, or from about 36 to about 48 hours. By using the term about, it is intended to mean that the viewing is performed within 30 minutes of said predetermined amount of time. This is useful so consistent images can be captured, particularly if the results of multiple viewings are to be compared to one another to see if the relative health of the skin and its hairs and follicles are improving, constant, or deteriorating.

In one embodiment, the shaving done prior to the viewing is performed using a razor having at least 3 blades, or at least 5 blades. Non-limiting examples of suitable multi-blade razors include disposable and system wet shave power or manual razors commercially available from Gillette®, such as under the Mach3®, Fusion®, or Fusion Proglide® product lines. In one embodiment, the shaving preparation step is also performed using a controlled shaving preparation (such as a shave foam or post foaming gel) as well as a set shaving regimen such that the skin appears to be clean shaven so no hairs are present protruding out of the portion of skin.

In one embodiment, the surface being viewed is on the neck or under the chin/jaw line. In another embodiment, the surface being viewed is on the face. In another embodiment, the surface being viewed can be any of the underarm, the arms, chest, back, legs, pubic area, scalp, and so forth. Those of skill in the art will appreciate that this method of viewing can be performed on any portion of skin, including those portions which have hairs. Further, as explained above, the skin need not be on a human as the method can also be performed with animals.

a. Angled Views

In one embodiment, the images are taken such that an angle between the first position to the focal point and the second position and the focal point is at least about 15°, or from about 15° and about 180°, or from about 30° to about 90°, or from about 45° to about 60°. Without intending to be bound by theory, it is believed that having such an angle between the first and second positions allows for sufficient separation of the positions to allow for a three dimensional view of the surface.

In one embodiment, a line drawn between the first position and the second position (and/or any subsequent positions) is not parallel to a plane tangential to the focal point. In another embodiment, a plane drawn between the first position, the focal point, and the second position is perpendicular to a plane tangential to the focal point. Where the surface is perfectly flat, a plane, tangential to the focal point, would be the plane of the surface. Where the surface is curved, such as convexly rounded, the plane, tangential to the focal point, is a plane that goes through that focal point and that it is tangent to that focal point.

A line drawn from the first position to the focal point can range from being parallel to a plane tangential to the focal point to being perfectly normal to that plane. In one embodiment, the first position is at an angle of from about 0° to about 90°, or from about 5° to about 45°, or from about 15° to about 30°, or from about 0° to about 15° from a plane tangential to said focal point. In certain instances, it may be preferred to have the first position be relatively close to the surface and then to have the second or subsequent image(s) be taken at higher angles from the plane, tangential to the focal point. This can be particularly useful if the images are taken along a same plane which is planar to the first position and the focal point.

In one embodiment, the second position or further subsequent positions is at an angle of from about 15° to about substantially perpendicular (or normal) to said tangential plane, or from about 15° to about 75°.

Without intending to be bound by theory, viewing the surface by taking multiple images allows for a three dimensioning of the surface along with any hairs or hair follicles there contained. It has been found that this can allow for enhanced diagnosis of skin conditions such as in grown hairs, obstructed hairs, as well as other dermatological conditions. Obstructed hairs, as referred to herein, means hairs which have grown beyond the surface of the skin and are pulling the skin immediately surrounding the hair away from the skin surface. In-grown hairs, on the other hand would be those hairs which are still contained below the surface of the skin and begin to curl and create bumps on the skin.

b. Focal Length—Distance from the Focal Point

In another embodiment, the first image is taken at substantially the same distance from the focal point as the second image. This distance is often times referred to as the focal length. In another embodiment, all images are taken at substantially the same distance such that they have substantially the same focal length. Substantially the same distance, as used herein, means that the shortest of the distances is within about 75% of the longest of the distances, preferably within about 90%, more preferably within about 95%, even more preferably within about 99%.

In another embodiment, the images can be taken at varying focal lengths. Where varying focal lengths are used, the distances of the viewing positions to the focal point should be recorded such that any subsequent analysis of the images taken can be correlated by geometry.

Those of skill in the art will understand that varying the angles and focal lengths can be done while still allowing the comparative views to provide useful information regarding the surface and any hairs or aberrations thereon. Aberrations on the surface, as referred to herein, can include skin irritations such as ingrown hairs, nicks and cuts, partially obstructed hair follicles, razor burn and so forth.

In one embodiment, the step of taking an image is taken at any focal length where a sufficiently clear image can be recorded. In one embodiment the focal length can be from about 0.1 cm to about 20 cm, preferably from about 1 cm to about 15 cm, more preferably from about 2 cm to about 10 cm.

c. Magnification

In one embodiment, one or more of the images are taken under a magnification greater than 1×. In one embodiment, at least one of the images is taken at a magnification of from about 2× to about 200×, preferably from about 3× to about 50×, more preferably from about 5× to about 25×. In one embodiment, all images are taken at the about same magnification, or exactly the same magnification. When using the term “about” in reference to magnification, it is mean that the degree of magnification is within an acceptable variation based on available equipment and use conditions. In one instance, “about” a degree of magnification can be within 0.1×. In another embodiment, one or more of the images can be taken at a different magnification from the other images.

Those of skill in the art will understand that varying the magnification can allow for more intricate analysis of the surface or any hairs under the surface, or protruding out of the surface. In some instances, it can be preferable to use a magnification higher than 20× so the actual hair, hair shaft, and/or follicles can be viewed in detail. As such, in one embodiment, the images are taken at a magnification of from about 20× to about 250×, or from about 50× to about 200×, or from about 75× to about 100×.

In other instances where a more topographical analysis is desired, the magnification can be below 20×. This relative lower degree of magnification can be useful to get a broader landscape view of the surface so bumps, crevices, ridges, and other aberrations in the skin surface can be viewed. This can be particularly useful in helping identify the number and severity of PFB and in grown hairs. In one embodiment, the images are taken at a magnification of from about 10× to about 20×.

d. Luminance

Those of skill in the art will understand that varying levels of luminance can be used as long as the images taken provide discernable surface contours and conditions as well as visible hairs and hair follicle positions and conditions. In one embodiment, said step of viewing said portion of skin is performed under a luminance of from about 1,000 candelas per square meter to about 500,000 candelas per square meter, preferably from about 5,000 candelas per square meter to about 250,000 candelas per square meter, more preferably from about 10,000 candelas per square meter to about 100,000 candelas per square meter.

In another embodiment, the level of luminance is substantially constant when taking the first image and when taking the second image, preferably within 1,000 candelas per square meter.

Further, imaging could also be conducted under different lighting set ups dependant on exactly what one wishes to view without deviating from the scope and intent of the present invention. For example, these varying lighting conditions include oblique lighting (i.e. from one side of the image field), imaging under blue light (could call out specific wavelengths of light), imaging under UV black light or other light wavelengths.

e. Taking the Multiple Images

In one embodiment, the step of taking said first image and said step of taking said second image are performed simultaneously. If additional images are also taken, they can also be taken simultaneously to the first image. This can be particularly useful to minimize environmental changes which can impact the surface, such as aging, lighting changes, temperature changes, movement, tightening or loosening of skin and or muscles, and so forth. As such, it may be preferable for all or at least the first few images to be taken at the same time, or within 1 minute, or within 10 seconds, or within 5 seconds, or within 1 second of one another.

In one embodiment, this method is performed such that an immediate identification of the skin condition can be provided to the subject being viewed. As such, it may be preferable to have the images taken as closely together in time as possible to minimize any variations which can undesirably impact the comparison of the relative images. In some cases, the goal could be considered to obtain multiple angled images of a specific area (or areas) of the skin surface to allow for a static three dimensional analysis of a said surface. In certain instances, it may be useful to perform the entire method several times so improvements to the health of the skin or the lack thereof can be identified so new treatment regimens can be pursued.

In one embodiment, the step of taking said images can be performed using the same viewing tool. Suitable viewing or imaging tools include microscopes, cameras and so forth, and are described in greater detail below. Where multiple images are taken using the same viewing tool, they can be taken at different times and positions by repositioning the surface relative to the viewing tool, or likely easier, by repositioning the viewing tool relative to the surface. In one embodiment, where multiple images are taken using the same viewing tool, they can be collected simultaneously.

f. Imaging Technologies

i. Microscopes

In one embodiment, the step of viewing said portion of skin comprises using a microscope comprising: a) housing having a proximal end and a distal end, said housing having an opening at the distal end; b) a sensor that receives light reflected from the surface of an object being viewed and converts said light to a digital image, the sensor being located at the proximal end of the housing, c) a lens that focuses light reflected from the object onto the sensor, the lens being located between the sensor and the opening; and d) at least one reflective member. In one embodiment the at least one reflective member can be located within the opening, wherein, in use, light is reflected from the object to the sensor and a portion of said reflected light is directed via the reflective member such that the surface of the object can be viewed simultaneously from two different directions. In one embodiment, the microscope can be digital. In another embodiment, the microscope can have more than one reflective members so multiple images can be captured at the same time. Non-limiting examples of suitable digital microscopes have been described in U.S. Patent No. 2011/0058030. Additional details of one such suitable microscope is provided in FIG. 9 and described below.

In another embodiment, a microscope without a reflective member can be used. In such an embodiment, the microscope can be used to take multiple images by being repositioned as needed. In another embodiment, multiple microscopes can be used. For example, if two images are taken, a different microscope can be used for each image.

Also suitable for use herein is confocal microscopy which is an optical imaging technique used to increase optical resolution and contrast of a micrograph by using point illumination and a spatial pinhole to eliminate out-of-focus light in specimens that are thicker than the focal plane. This technique enables the reconstruction of three-dimensional structures from the obtained images.

Stereo microscopy can also be used. Stereo microscopy, in contrast to confocal microscopy uses incident light illumination rather than transillumination. It uses two separate optical paths with two objectives and two eyepieces to provide slightly different viewing angles to the left and right eyes. In this way it produces a three-dimensional visualization of the sample being examined.

The basic concepts of confocal microscopy and stereo microscopy can also be applied to other imaging technologies described below such that cameras or video recorders can be modified to have confocal viewing or stereo views.

ii. Still Cameras

In one embodiment, the image(s) can be taken using one or more still cameras, which can be selected from digital cameras, film cameras, acoustic cameras, optical cameras, or combinations thereof. If using film, it may be useful to scan the resultant image into a computer if a computer analysis is desired.

In one embodiment, the images are taken from a stereo imaging camera. The camera can be an SLR camera or a typical USB camera such as those used with desktop and laptop computers. In another embodiment, the camera can be on a hand held device such as a cell phone or smart phone. Where the camera is on a smart phone, an application can be provided to the user so they can view a portion of skin and immediately use the application to determine the condition of the skin. As with any of the imaging technologies, the camera can have a single view or can have a reflective member to allow for stereo imaging. Further, a device can be provided which controls multiple cameras or other imaging devices such that the set up can position the imaging devices and allow them to take images at the same time, in series, under the same lighting conditions (i.e. where each camera lens has its own flash set at the same relative angle, and so forth).

Various types of stereo imaging cameras are known and can be used in accordance with the present invention. See, e.g., U.S. Pat. No. 6,701,081 for a dual camera mount for stereo imaging; US Pubs. 2010/0283833 for a digital image capturing device with stereo image display and touch functions, having a plurality of image capturing units with a module; 2006/0077543 for a stereo camera having a stereo optical module which acquires a stereo image; a central control unit which evaluates the object as a target based on the stereo image acquired by the stereo optical module; and a communication line through which the stereo image data acquired by the stereo optical module is input to the central control unit; and 2002/0158984, disclosing a stereo camera system comprising: a stereo imaging means for outputting at least one stereo image; recognition means for locating an object of interest in the field of view of the stereo imaging means and at least one of a distance of the object of interest from the stereo imaging means and the size of the object of interest; and adjusting means for automatically changing at least one system parameter which affects the spatial resolution of the object of interest based on at least one of the located distances of the object of interest from the stereo imaging means and the size of the object of interest.

iii. Video Imaging

In another embodiment, stereo video can be used to image the surface. Similar to using still images, a video recording system can be used to capture video of the surface and focal point from various view angles and/or focal distances. This can be particularly useful if the analysis of the surface requires recording of movement of the surface relative to an external stress such as applied pressure from touching, or temperature changes, or lighting changes, and so forth. Without intending to be bound by theory, it is believed that stereo video imaging can be useful if viewing the elasticity of the surface. Also useful could be to view formation of goose bumps or other skin change phenomenon. All of these and other phenomenon may be suitable for stereo video imaging based on the desired analysis.

iv. Manual Viewing

In another embodiment, the surface can be viewed by the human eye, preferably assisted with a viewing lens, such as a magnifying glass or jeweler's scope. In such an embodiment, the viewer can note various factors such as length of hairs, occurrence of raised skin pulps, obstructed hair follicles, in grown hairs, and so forth. The user can then input these pieces of information into a computer for subsequent analysis, or they can analyze the data manually to provide a resultant analysis of the surface having been viewed.

2. Analysis Step

As explained above, the step of viewing said surface can result in various pieces of information being captured, recorded, or otherwise calculated. Those of skill in the art will understand that the type of information desired will be based in part on the type of analysis being conducted. For example, if one were to analyze the coloration of the skin surface, the light reflectance levels of the surface would be useful to know. Non-limiting examples of useful types of information are described throughout the present application and also include, number of hairs protruding from the skin surface, thickness of hairs, diameter or cross sectional shape of hairs, length of hairs protruding from the surface, any topographical changes on the skin surface such as bumps or crevices; in grown hairs, obstructed hairs which result in raised portions of skin; clogged pores, and so forth.

In one embodiment, the method further comprises a step of analyzing said portion of skin comprising: recording the number of hairs present in said portion of skin; determining the average length of said hairs; and calculating the percentage of hairs which is less than a certain percentage of the average length, for example less than about 25%, or less than about 30%, or less than 50%, or less than 75%. The step of determining the average length of said hairs could include a step of determining the length of each hair present in the images, then taking the average of said lengths. The length of hair, as described herein, would be measured from the tip of the hair to the farthest reaching portion of skin surrounding said base of the hair, along the length of the hair. The length of said hairs can be determined by a computer using an algorithm to determine the length based on the various images recorded. The algorithm can rely on basic geometry based on the positioning with which the images or taken. The lengths can also be calculated by hand if so desired. In one embodiment, a computer can calculate the length and numbers of hairs, the average length of hairs, as well as the percentage of hairs within a set length of the average length. This program can then output a single measurement index to describe the condition of said portion of skin.

Depending on the percentage of hair which are determined to be below a certain length with respect to the average length, a rating can be assigned to that portion of skin. For example, if a portion of skin on a subject's neck has 5% to 25% of hairs less than 50% of the average hair length, then that portion of skin can be considered to be rich in in-grown hairs and/or obstructed hairs. This region of the skin can be in need of specific treatment to minimize discomfort when shaved or otherwise treated.

In another embodiment, it may be useful to focus on either the number of in-grown hairs (occurrences of PFB) and/or number of obstructed hairs. This form of analysis can be done manually or via a computer algorithm and includes the steps of: determining the number of obstructed hair and/or in grown hairs present in the portion of skin having been viewed; determining the number of hairs present in said portion of skin; and calculating the percentage of obstructed hairs and/or in grown hairs. Based on this percentage, a rating of skin/hair health can be provided. In another embodiment, other skin heath conditions can be measured and analyzed, such as occurrence of nicks and cuts, pock marks, scars, discolorations, acne, and so forth.

Another suitable step of analysis can include 1) identifying a hair by the difference in color of the hair compared to surrounding skin; 2) identifying differences in hair color along the length of the hair (above and/or below the surrounding skin); and determining whether this hair is considered obstructed based on the relative amount of hair above the surrounding hair versus the average of the other hairs.

Yet another type of analysis can include: identification of localized mounding of skin around the base of a hair or hair follicle. This type of topographical analysis could be used to determine PFB, obstructed hairs, clogged pores, acne, insect bites, puss filled pores, rashes, bacterial or fungal infections, or a multitude of other skin conditions or irritations. A similar “color” analysis step comparing any portion of hair above vs. below the surrounding skin could be used. Further, color imaging or x-ray imaging can be used to determine the presence of hairs trapped under the skin. The length of the hairs as well as the curvature under the skin can be recorded and analyzed to determine the occurrence of PFB or such conditions.

3. Prescribing Step

In one embodiment of the present invention, the method further comprises a step of prescribing a treatment regimen for the analyzed surface. Various types of skin treatment compositions and protocols have been described in the art for various skin ailments or conditions.

Without intending to be bound by theory, it is believed that based on the analysis, certain skin conditions can be identified and therefore paired up with recommended treatment regimens in a prescribing step. For example, if a portion of skin is found to have a certain propensity for PFB or obstructed hairs, it may be useful to prescribe to them razor blade that has 5 or more blades. Or the user can be instructed to shave more regularly using specific shaving treatments such as shaving foams or gels, and hot water, for set amounts of time. In another embodiment, if the surface is found to have a relatively high occurrence of abrasions such as nicks and cuts, it may be useful to prescribe a more efficient shaving razor, or the a shaving preparation which has higher lubrication with specific lubrication polymers to decrease any irritations that may occur from shaving with less advanced shaving technologies. Other skin conditions can include excessive wrinkling, discoloration, acne, pock marks, and so forth. Various types of treatment products and regimens have been described for all kinds of these conditions and can be used in a prescribing step to advise the subject on how to treat their conditions.

4. Methods of Use

a. Point of Sale Diagnostic

In one embodiment, the method of the present invention is used in a store setting. A subject can enter the store and sit at a diagnostic kiosk. The method of viewing, analyzing and prescribing a treatment can be performed while the subject is present. In another embodiment, the step of viewing can be done with the subject present and the other steps can be performed thereafter with or without the subject present. The analysis and recommendation can then be provided to the subject via a separate communication or at a subsequent visit. In certain instances, it may be useful to do the entire process with the subject present so a treatment step can be conducted by a treatment professional. Additionally, free samples of treatment products can be provided to the subject to help them understand the specific treatments recommended via the present method based on their specific skin conditions. Those of skill in the art will understand that the number of skin and hair treatment products and protocols on the market can be near impossible to estimate. It is believed that this tool, allows for a method of viewing a surface that can allow for a more accurate analysis and diagnosis. It is further believed that this will help consumers cut through the ocean of products marketed at them so they can use a product which is specifically recommended for their specific skin/hair type and condition.

b. At Home/Point of Use Diagnostic

In another embodiment, the method can be conducted by the subject themselves using a hand held or otherwise easy to use imaging tool. The device can also perform the analysis step, or the image(s) and respective data can be uploaded to a central analysis module which performs the analysis, then sends back the recommendation. This can be done via a user terminal (i.e. a home computer or hand held device) for the viewing step, then rely on the internet to send the data back to a server which performs the analysis and subsequent steps then sends back down to the terminal to inform the subject of the results and recommendation. Providing an at home system to perform the present method of diagnosis can be very desirable for many consumers as it can be less time consuming and more private of a diagnosis. It is believed that some consumers may not want to go to a facility to have their skin diagnosed by a stranger. By providing an at home tool, the present invention allows for users to get the attention that they need to properly diagnosis and treat their skin all while in the comfort of their own home. Furthermore, when a recommendation is provided to the subject, they can simply approve the recommendation and place an order to a desired location with just the click of a button.

As explained above, where the camera is on a home computer or hand held device such as a smart phone, an application can be provided to the user so they can have the viewing step, the analysis step, and a recommendation step performed all via one tool. The application can be downloaded onto the device, or can be made available over the internet. As explained above, a further step of ordering any recommended products or treatment guides can also be performed either with the same device in the same session, or thereafter at the subjects' convenience.

When allowing subjects to perform the viewing step on their own, it can be important to ensure that the images are taken in the right conditions. When the method is conducted in store, a consultant or technician can be available to ensure proper set up and execution of the steps. When at home, certain safeguards may be desirable to help minimize user error and ensure more reliable images. In one embodiment, the device used to capture the images has a distance setting member to ensure the focal distance is properly set and then recorded for later analysis. The distance setting member can be something simple like a stand upon which the camera is positioned such that it sits a set distance from the focal point. The camera can then be set to focus on a specific point. Angling and/or movement of the stand would then change the distance and/or angle but allow the user to more easily ensure they are using the device and performing the steps as recommended. In another embodiment, where the camera is in a fixed position (such as a USB camera on a laptop, a head harness can be used to position the head a set distance from the camera lens. The harness can allow for the head or other body part to be repositioned so various angled images of the same focal point can be captured.

Also important would be for the amount of relative lamination to be controlled or otherwise recorded when the images are being captured. Without intending to be bound by theory, it is believed that controlling or accounting for lamination conditions can also be important to ensure the quality of images is such that a useful analysis and subsequent recommendation can be provided.

5. Figures

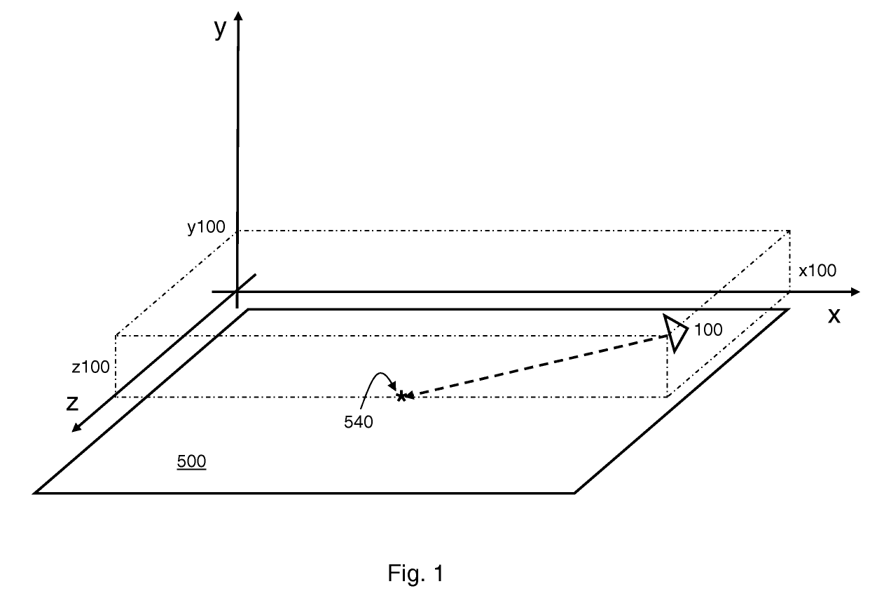

Turning to the figures, FIG. 1 shows a surface being viewed from a first position 100.

FIGS. 1-8 are elevated perspective views of various embodiments of the present invention wherein a surface is viewed from one or more positions. Shown in each of these figures is a three dimensional Cartesian coordinate system with X, Y and Z axes. The purpose of all of these views and positions is to show the relative placement of the first and second positions.

FIG. 1 shows a surface 500 with a focal point 540 thereon. The first position 100 is present at coordinates x100, y100 and z100. FIG. 1 further shows a second position 200 at x200, y200, and z100. As shown in this figure, the first position and the second position are aligned such that they form a plane intersecting the focal point. This plane can be perpendicular to the plane tangential to the focal point. Further this plane can be parallel with a line normal to the focal point. Angle 610 is the angle along this plane formed by lines drawn from the first position to the focal point and the second position and the focal point. This angle can be referred to as the z-plane angle. As shown in these figures, the focal distances are the same for any positions on the same figure.

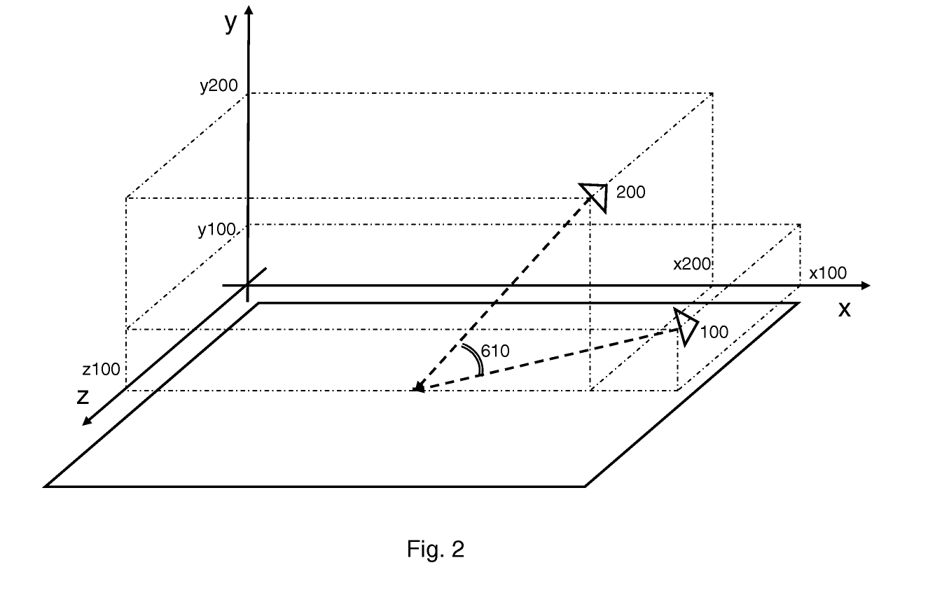

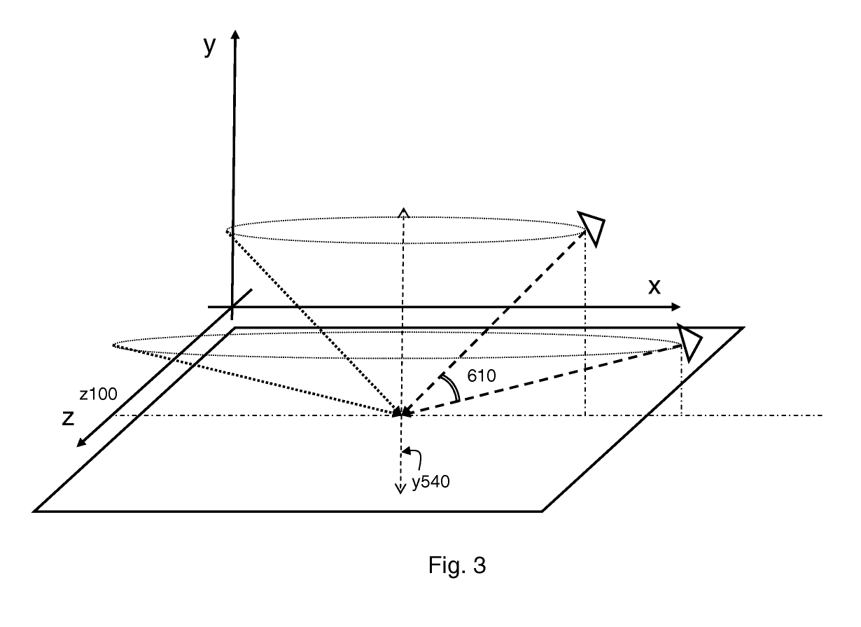

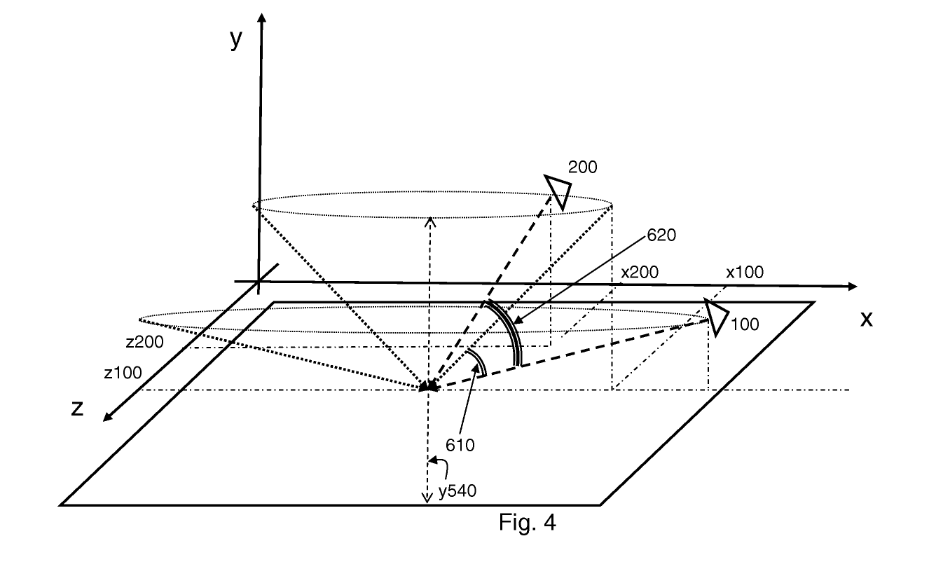

FIG. 3 is similar to FIG. 2 in that a first position and a second position are both taken along z100. The first position and the second position are swung around 180° about a line y540 which is parallel to the y-axis and drawn through the focal point. In this example, the z-plane angle is shown as 610. FIG. 4 shows another embodiment, where the second position is swung about the y540 line such that it now resides along z200. z-plane angle 610 would be measured by brining the second position back until it is on the same z position as the first position. Absolute angle 620 would be measured as the actual angle measured between lines extending from the focal point to the first position, and from the focal point to the second position. For purposes of clarification, those of skill in the art will understand that the absolute angle need not be the angle measured along any given plane passing through a single point along the x, y or z axes. The absolute angle, can however be the same as one of these angles, such as in FIG. 3, where the absolute angle would be the same as the z-plane angle.

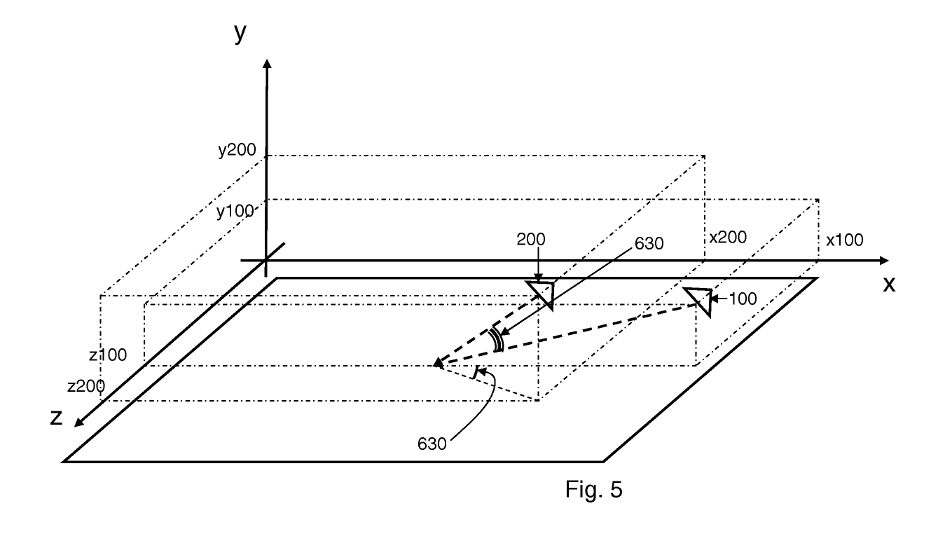

FIG. 5 shows yet another alignment of the first and second positions. In this figure, z200 is greater than z100. A yO-plane angle is shown as 630. The yO-plane is determined as the angle between two lines taken along an x-z plane along the 0 position of the y axis. Those of skill in the art will understand that x0 and z0 plane angles can also be measured.

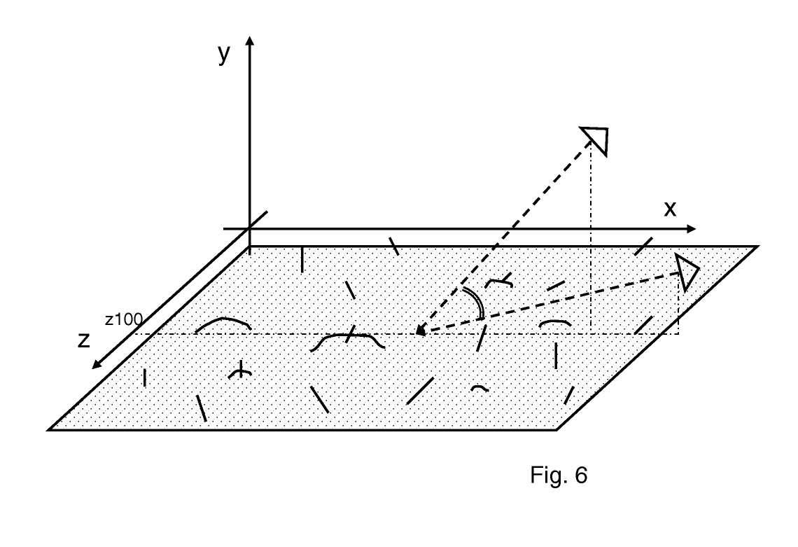

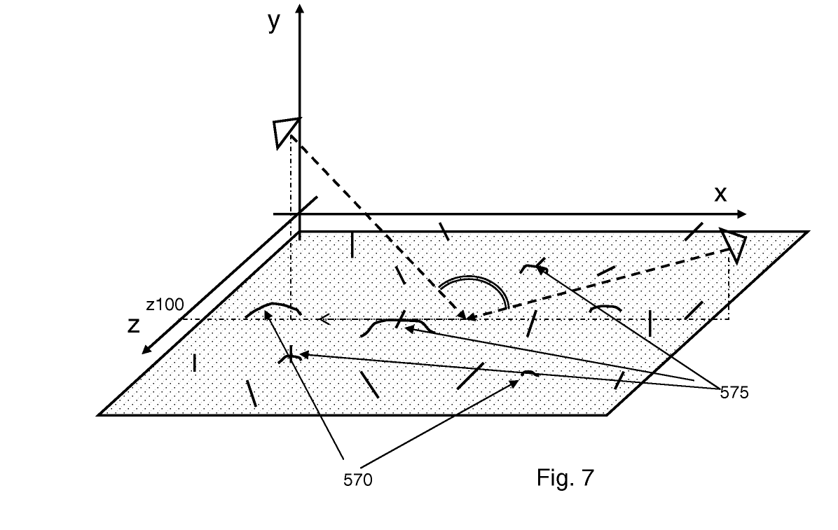

FIGS. 6 and 7 show two other placements of the second position such that in FIG. 6 an acute angle is formed along the z100-plane, while an obtuse angle is formed along the z-100 plane. The z-plane angle in FIG. 7 would be acute (where the second position would be rotated around an y540 line by 180°. FIGS. 6 and 7 also show hairs which protrude out of the skin as well as in grown hairs (razor bumps) 570 and obstructed hairs which are growing out of similar extensions of skin 575.

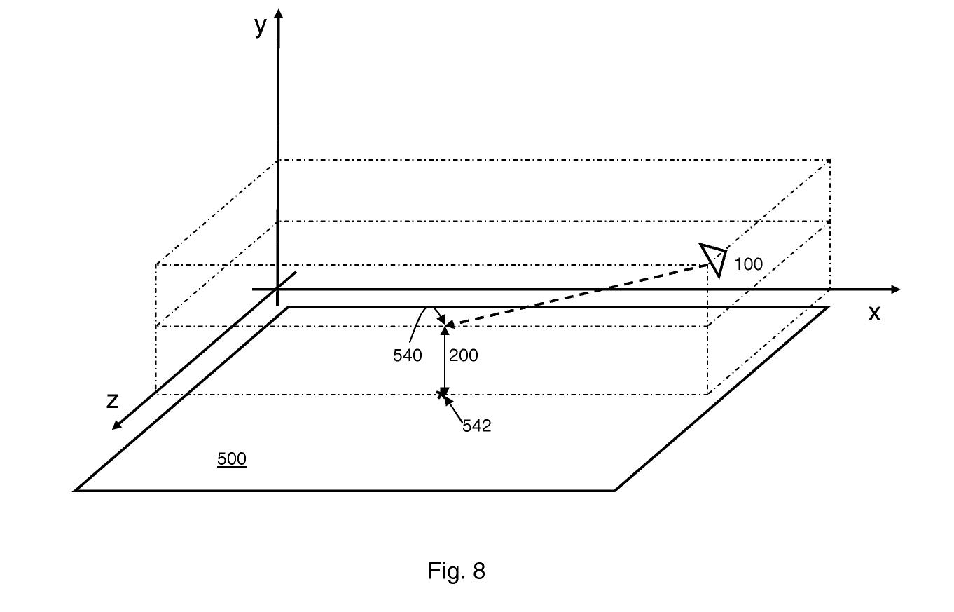

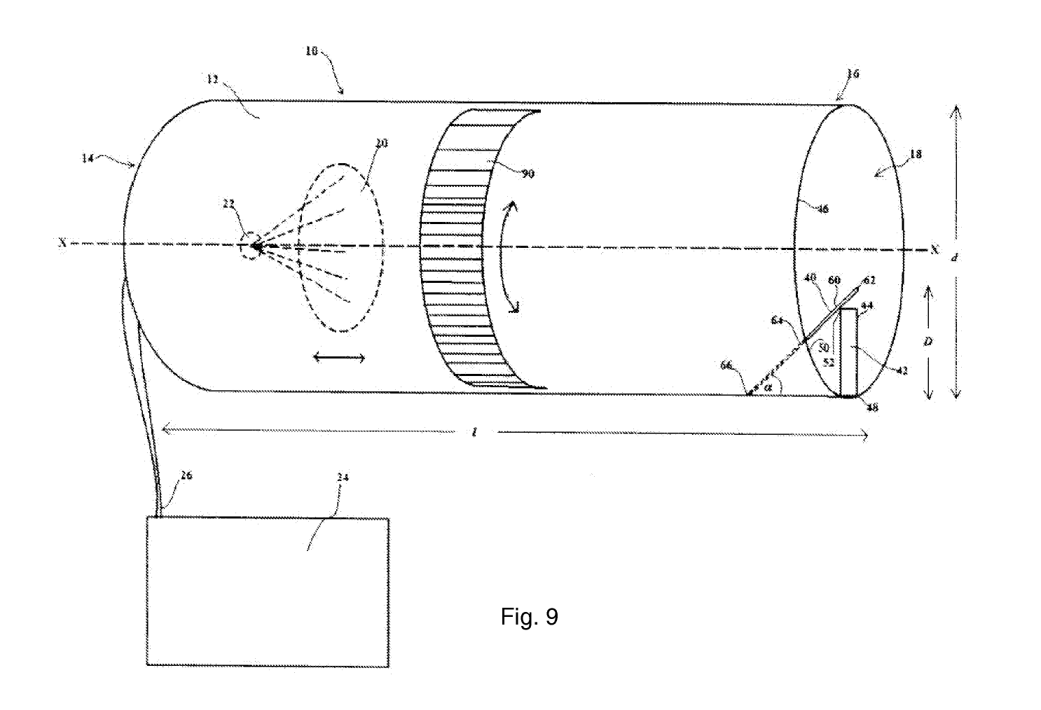

FIG. 8 shows another embodiment, where the focal point resides a set distance above the surface. Distance 200 is measured as straight line distance from the focal point to the point there under the surface. In this situation the focal point 540 resides a distance 200 above the surface, directly above surface focal point 542.

FIG. 9 shows a perspective view of a digital microscope 10 formed of a housing 12 having a proximal end 14 and a distal end 16. The distal end 16 forms an opening 18 to the housing 12. In use, the opening 18 is pressed against or placed near objects intended for viewing. A lens 20 is located within the housing, perpendicular to a length, 1, of the housing 12. The lens 20 is movable between the proximal 14 and distal 16 ends of the housing 12 to allow a user to focus on an object to be viewed. A sensor 22 is provided between the lens 20 and the proximal end 14 of the housing. In use, light is reflected from the surface of an object to the lens 20 and subsequently focused on the sensor 22. The sensor 22 is preferably a charge-coupled device (CCD) that captures the reflected light and converts it to digital data that is recorded for the microscope, in the manner of a camera. A resulting image is shown on an associated screen 24. This screen 24 may be directly attached to the microscope 10 (not shown) or the microscope 10 may have a Universal Serial Bus (USB) connection 26 such that the image may be viewed on a computer screen 24.

A reflective member 40 is secured to a projection 42 that is located within the opening 18 of the housing. The projection 42 shown in FIG. 9 has a first side 44 in line with an edge 46 of the opening 18, a second side 48 adhered to an internal surface 50 of the housing 12, and a third side 52 to which the reflective member 40 is adhered. The projection shown in FIG. 9 is rectangular in shape. However, it will be appreciated that any form of projection 42 or securing member can be used to secure the reflective member 40 in the desired position. For example, the projection 42 shown in FIG. 2 is triangular in shape. Furthermore, it will be appreciated that the projection 42 may be formed integrally with the housing or secured to an internal surface of the housing in a known manner.

In the embodiment shown in FIG. 9, the reflective member 40 is a mirror 60. The mirror 60 is fixed to the third side 50 of the projection. A reflective surface 64 of the mirror has a first edge 66 located adjacent the internal surface 50 of the housing and a second edge 62 substantially in line with the edge 46 of the opening 18. It will be appreciated that the mirror 60 could extend beyond the edges of the projection 42. The reflective surface 64 of the mirror 60 is positioned at an angle, α, of between 30°, 35° or 40° and 50°, 55° or 60° to the internal surface 50 of the housing 12. In a particular embodiment, the mirror 60 is positioned at an angle a of 45° to the internal surface 50 of the housing 12. The second edge 62 of the mirror 60 is positioned away from the internal surface 50 of the housing to which the projection is attached at a distance of between 30%, 35%, 40% or 45% and 55%, 60%, 65% or 70% of the diameter, d, of the opening end of the housing. In a particular embodiment, the second edge 62 of the mirror 60 is positioned a distance of 50% of the diameter, d, of the opening of the housing away from the internal surface 50 of the housing 12 to which the projection 42 is attached.

The mirror 60 shown in FIG. 9 is a first surface mirror. First surface mirrors have the reflecting surface placed on the front or first surface of the glass to eliminate internal reflection. In the present application, use of a first surface mirror prevents ghosting of the image as seen on screen. It will, however, be appreciated that other mirror types may be used to achieve a similar effect.

The lens 20 shown in FIG. 9 is positioned substantially perpendicular to a longitudinal axis of the housing such that the centre of the lens coincides with a point on the longitudinal axis. The sensor 22 is located between the lens 20 and the proximal end 14 of the housing at some point along the longitudinal axis in line with the centre point of the lens 20. It will, however, be appreciated that the relative positions of the lens and sensor could be different if the lens is arranged to focus onto a different position. Furthermore, multiple sensors and/or lenses could be provided. For example, multiple lenses may be used to provide multiple magnification levels/points. In embodiments, the lens may be bi-focal to ensure that both parts of the image are kept in focus. Additionally, multiple sensors may be used, for example, to detect different colors in the images.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm” All measurements are performed at 25° C., unless otherwise specified.

All documents cited in the DETAILED DESCRIPTION OF THE INVENTION are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern. Except as otherwise noted, the articles “a,” “an,” and “the” mean “one or more.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of viewing a portion of surface, optionally having at least one hair, comprising:
a. taking a first image of a focal point of said portion of skin from a first position,
b. taking a second image of said portion of skin from a second position,
c. wherein an angle between the first position to the focal point and the second position and the focal point is at least about 15°, and
d. wherein the first image is taken at the same focal length from the focal point as the second image.

2. The method of claim 1, wherein the angle is from about 15° and about 180°.

3. The method of claim 2, wherein the plane drawn between the first position, the focal point and the second position is perpendicular to a plane tangential to the focal point.

4. The method of claim 1, wherein the first position is at an angle of from about 0° to about 15° from a plane tangential to said focal point.

5. The method of claim 4, wherein the second position is at an angle of from about 15° to about substantially perpendicular to said tangential plane.

6. The method of claim 1, wherein a line drawn between the first position and the second position is not parallel to a plane tangential to the focal point.

7. The method of claim 1, wherein said step of taking said first image is taken at a focal length of from about 0.1 cm to about 20 cm, preferably from about 1 cm to about 15 cm, more preferably from about 2 cm to about 10 cm.

8. The method of claim 1, wherein the focal point is at the surface of said portion of skin to a distance of about 1 cm above said portion of skin.

9. The method of claim 1, wherein said step of viewing said portion of skin is performed under a luminance of from about 1,000 candelas per square meter to about 500,000 candelas per square meter, preferably from about 5,000 candelas per square meter to about 250,000 candelas per square meter, more preferably from about 10,000 candelas per square meter to about 100,000 candelas per square meter.

10. The method of claim 9, wherein the level of luminance is substantially constant when taking the first image and when taking the second image, preferably within 1,000 candelas per square meter.

11. The method of claim 1, wherein taking said first image is performed at a magnification of from about 2× to about 200×, preferably from about 3× to about 50×, more preferably from about 5× to about 25×.

12. The method of claim 11, wherein said step of taking said second image is performed at the same magnitude as the step of taking said first image.

13. The method of claim 1, wherein said step of taking said first image and said step of taking said second image are performed simultaneously.

14. The method of claim 1, wherein said step of taking said first image and said step of taking said second image are performed using the same microscope.

15. The method of claim 14, wherein the step of taking said first image and said step of taking said second image are performed simultaneously and comprises using a digital microscope using confocal microscopy or stereo microscopy.

16. The method of claim 1, wherein said portion of skin is on the neck or under the chin/jaw line.

17. The method of claim 1, wherein said step of viewing said portion of skin comprises taking a third image.

18. The method of claim 1, further comprising a step of:
a. analyzing said portion of skin comprising:
i. recording the number of hairs present in said portion of skin;
ii. determining the average length of said hairs; and
iii. calculating the percentage of hairs which is less than about 25% of the average length.

19. The method of claim 18, wherein said step of determining the average length of said hairs is determined by an algorithm.

20. The method of claim 1, further comprising a step of:
a. analyzing said portion of skin comprising:
i. determining the number of obstructed hair present in said portion of skin;
ii. determining the number of hairs present in said portion of skin; and
iii. calculating the percentage of obstructed hairs.
